(19) United States
(12) Patent Application Publication
(10) Pub. No.: US 2018/0207331 A1
(43) Pub. Date: Jul. 26, 2018

(54) CHAMBER FOR THE COLLECTION OF LIPOASPIRATE

(71) Applicant: Puregraft LLC, Solana Beach, CA (US)

(72) Inventors: Bradford A. Conlan, Solana Beach, CA (US); Lucas Fornace, Encinitas, CA (US); Thomas Fuller, Dubuque, IA (US)

(21) Appl. No.: 15/411,400

(22) Filed: Jan. 20, 2017

(51) Int. Cl. A61M 1/00

(52) U.S. Cl. CPC A61M 1/0056; A61M 1/0017; A61M 2202/0014; A61M 2202/08; A61M 1/0003

(57) ABSTRACT

Embodiments of the present invention disclose a device for collection and purification of adipose tissue and methods of making and using the same.

### FIELD OF THE INVENTION

The invention relates generally to biomedical sciences and technologies and particularly to tissue harvesting and tissue graft application devices and methods.

### BACKGROUND OF THE INVENTION

The transfer of adipose tissue to various regions of the body is a relatively common cosmetic, therapeutic and structural procedure involving the harvest of adipose tissue from one location and re-implantation of the harvested and, oftentimes processed tissue, in another location (see Coleman 1995; and Coleman 2001). While being largely used for repair of small cosmetic defects such as facial folds, wrinkles, pock marks and divots; the transfer of adipose tissue has recently been used for cosmetic and/or therapeutic breast augmentation and reconstruction (Bircoll and Novack 1987; and Dixon 1988), and augmentation of the buttocks (Cardenas-Camarena, Lacouture et al. 1999; de Pedroza 2000; and Peren, Gomez et al. 2000).

In the past, adipose tissue grafts and methods of adipose tissue transfer have been plagued with difficulties and side effects including necrosis, absorption of the implant by the body, infection (Castello, Barros et al. 1999; Valdatta, Thione et al. 2001), calcifications and scarring (Huch, Kunzi et al. 1998), inconsistent engraftment, (Eremia and Newman 2000), lack of durability, and other problems arising from lack of neovascularization and necrosis of the transplanted tissue. One of the biggest challenges in adipose tissue transfer is absorption of the implant by the body and volume retention of adipose tissue grafts following transfer. When adipose tissue is harvested or washed, the space between individual pieces of harvested adipose tissue is filled by liquid (e.g., water, blood, tumescent solution, oil). When this tissue/fluid mixture is implanted into a recipient the liquid portion is rapidly absorbed by the body resulting in loss of volume. The process by which the amount of fluid is removed from the tissue/fluid mixture is frequently referred to as “drying the adipose tissue” or “dehydrating the adipose tissue”. The content of red and white blood cells and the like within an adipose tissue graft can also significantly affect the volume of graft retained after graft transplantation, due to induction or exacerbation of an inflammatory response. Another aspect of tissue retention relates to the amount of lipid within the adipose tissue graft. It understood that the presence of free lipid (meaning lipids released from dead or damaged adipocytes; also referred to as oil) in adipose tissue grafts can result in induction or exacerbation of an inflammatory response with substantial phagocytic activity and consequent loss of graft volume.

It is also known that mixing unprocessed adipose tissue with a concentrated population of adipose-derived regenerative cells overcomes many of the problems associated with adipose tissue grafts and adipose tissue transfer, as described above. Specifically, supplementing unprocessed adipose tissue with concentrated populations of adipose-derived cells comprising adipose-derived stem cells increases the weight, vascularization, and retention of fat grafts. (See U.S. Pat. No. 7,390,484 and co-pending U.S. Patent Application Publication No. 2005/0025755, issued as U.S. Pat. No. 7,651,684, herein expressly incorporated by reference in their entireties). Adipose tissue fragments supplemented, or mixed, with a concentrated population of cells including adipose-derived stem cells exhibit improved neoangiogeneis and perfusion in grafts when compared to unsupplemented grafts of adipose tissue alone in animal models. Further, adipose tissue grafts supplemented with adipose-derived regenerative cells that comprise adipose derived stem cells show increased graft retention and weight over time, when compared to unsupplemented grafts. (See U.S. Patent Application Publication No. 2005/0025755, issued as U.S. Pat. No. 7,651,684). Further, the processing of adipose tissue in a closed, sterile fluid pathway greatly reduces the chance of infection. The improvement in autologous transfer of adipose tissue seen in the animal models described above has also been replicated in human clinical studies. Nevertheless, the isolation and purification of concentrated populations of adipose-derived regenerative cells comprising adipose-derived stem cells (ADSCs), usually involves a series of washing, digestion, filtration and/or centrifugation steps, which can reduce the yield of viable cells, require mechanical equipment and specialized clinicians, and/or can compromise the quality, appearance, longevity, hydration or efficacy of the graft.

Additionally, stresses could cause undesirable reactions to harvested adipose tissues. Such stresses include, for example, exposure to environmental pathogens, which are mentioned above, and prolonged post-harvest storage, etc. Therefore, there is a need for in-situ harvest, cleaning, and use of an adipose tissue graft for biomedical applications.

The current methods of collecting, preparing, and delivering fat tissue grafts typically suffer from one or more of the following issues:

a) requiring one device to collect tissue, and another to process;

b) being prone to tipping over;

c) incapable of fully drying the tissue to the desired consistency;

d) having many components that need to be sterilized ahead of time;

e) having numerous connections to be made that can be confusing;

f) having limited tissue volume capacity;

g) having tissue that gets stuck or is difficult to fully remove (leading to loss);

h) being open to the environment (not a true “closed-system”)

The need for additional approaches to prepare and optimize adipose tissue grafts and implants and to isolate and/or concentrate adipose-derived regenerative cells is manifest.

### SUMMARY OF THE INVENTION

In one aspect of the present invention, it is provided a device for adipose tissue collection and purification, comprising:

a) an adipose tissue collection and filtration component on the inside;
b) a rigid container (“canister”) on the outside that allows for a vacuum collection of lipoaspirate; and
c) a lid on the rigid container comprising a fluid logic,

wherein the fluid logic is controlled by a pinch cam which selectively pinches off tubing connections depending on the desired fluid configuration, and

wherein the fluid logic comprises a configuration such that when there is a need to open the canister, the adipose tissue collection and filtration component and lid remain in connection and closed to the atmosphere.

In some embodiments of the invention device, optionally in combination with one or more embodiments of invention disclosed herein, the entire assembly is pre-assembled and disposable.

In some embodiments of the invention device, optionally in combination with one or more embodiments of invention disclosed herein, the entire assembly is made entirely of plastic.

In some embodiments of the invention device, optionally in combination with one or more embodiments of invention disclosed herein, the fluid connections/logic is controlled by the product itself as opposed to humans needing to make and adjust connections.

In some embodiments of the invention device, optionally in combination with one or more embodiments of invention disclosed herein, the lid comprises a knob,

wherein the pinch cam comprises pitch valves, and

wherein turning of the knob causes the pinch valves to open and/or close in accordance with the fluid logic diagram.

In some embodiments of the invention device, optionally in combination with one or more embodiments of invention disclosed herein, the fluid logic comprises a plurality of ports and positions related to each other in a way to effect adipose tissue collection and purification.

In some embodiments of the invention device, optionally in combination with one or more embodiments of invention disclosed herein, the position comprises a harvest position, a wash position, and an off position, wherein:

in the “harvest” position, the fluid logic would be such that a vacuum source via a vacuum port would be pulling tissue from a patient port and into the adipose tissue collection and filtration component, in the meanwhile pulling fluid out of a “drain” port;

in the wash position, the drain would be closed off so the adipose tissue collection and filtration component is filled with fluid; and

in the off position, all of the pinch valves are closed and an “eccentric” cam is used to eject the lid off to open it and gain easy access to the adipose tissue collection and filtration component inside in the event that some of the tissue is stuck in the adipose tissue collection and filtration component to squeeze out the stuck tissue.

In some embodiments of the invention device, optionally in combination with one or more embodiments of invention disclosed herein, the adipose tissue collection and filtration component is a Puregraft™ bag.

In another aspect of the present invention, it is provided a method of adipose tissue collection and purification, comprising:

providing a device comprising
a) an adipose tissue collection and filtration component on the inside;
b) a rigid container (“canister”) on the outside that allows for a vacuum collection of lipoaspirate; and
c) a lid on the rigid container comprising a fluid logic, wherein the fluid logic is controlled by a pinch cam which selectively pinches off tubing connections depending on the desired fluid configuration, and wherein the fluid logic comprises a configuration such that when there is a need to open the canister, the adipose tissue collection and filtration component and lid remain in connection and closed to the atmosphere, and

performing adipose tissue collection and purification using the device so as to harvest and purify an amount of adipose tissue in a manner that the adipose tissue collection and filtration component and lid remain in connection and closed to the atmosphere.

In some embodiments of the invention method, optionally in combination with one or more embodiments of invention disclosed herein, the entire assembly of the device is pre-assembled and disposable.

In some embodiments of the invention method, optionally in combination with one or more embodiments of invention disclosed herein, the entire assembly of the device is made entirely of plastic.

In some embodiments of the invention method, optionally in combination with one or more embodiments of invention disclosed herein, the fluid connections/logic is controlled by the product itself as opposed to humans needing to make and adjust connections.

In some embodiments of the invention method, optionally in combination with one or more embodiments of invention disclosed herein, the lid comprises a knob,

wherein the pinch cam comprises pitch valves, and

wherein turning of the knob causes the pinch valves to open and/or close in accordance with the fluid logic diagram.

In some embodiments of the invention method, optionally in combination with one or more embodiments of invention disclosed herein, the fluid logic comprises a plurality of ports and positions related to each other in a way to effect adipose tissue collection and purification.

In some embodiments of the invention method, optionally in combination with one or more embodiments of invention disclosed herein, the position comprises a harvest position, a wash position, and an off position, wherein:

in the “harvest” position, the fluid logic would be such that a vacuum source via a vacuum port would be pulling tissue from a patient port and into the adipose tissue collection and filtration component, in the meanwhile pulling fluid out of a “drain” port;

in the wash position, the drain would be closed off so the adipose tissue collection and filtration component is filled with fluid; and

in the off position, all of the pinch valves are closed and an “eccentric” cam is used to eject the lid off to open it and gain easy access to the adipose tissue collection and filtration component inside in the event that some of the tissue is stuck in the adipose tissue collection and filtration component to squeeze out the stuck tissue.

In some embodiments of the invention method, optionally in combination with one or more embodiments of invention disclosed herein, the adipose tissue collection and filtration component is a Puregraft™ bag.

### BRIEF DESCRIPTION OF THE DRAWINGS

FIG. 1 illustrates an embodiment of the invention device.

FIG. 2 is a photographic image of a 3D-printed/machined prototype of lid 300 with the liquid logic diagram 310.

FIG. 3 shows an invention device 100 being tested along with a pump 410 (left) and catch canister 420 (center).

### DETAILED DESCRIPTION OF EMBODIMENTS OF THE INVENTION

### Definitions

As used herein, the term “adipose tissue” is used interchangeably with the term “fat”, the meaning of which is well known to a person of ordinary skill in the art.

As used herein, the term “adipose tissue removal” or “tissue removal” or “harvesting” or “liposuction” are used interchangeably to mean remove an amount of adipose tissue from a live subject such as a male or female patient.

As used herein, the term “Puregraft™ bag” refers to an adipose tissue collection and purification bag marketed by Puregraft LLC. Examples of Puregraft bag are described in U.S. Pat. No. 9,133,431 B2, teaching of which is incorporated herein by reference. In some embodiments, “Puregraft™ bag” is used interchangeably with the term “PG bag”.

As used herein, the term “filter”, if applicable, refers to a porous material having a size or size distribution useful for adipose tissue filtration. Some examples of filter useful for the present invention are described in U.S. patent application Ser. No. 12/771,985, issued as U.S. Pat. No. 9,133,431, the teachings of which is incorporated herein by reference in its entirety.

### Adipose Tissue Collection and Purification

In one aspect of the present invention, it is provided a device for adipose tissue collection and purification, comprising:

a) an adipose tissue collection and filtration component on the inside;
b) a rigid container (“canister”) on the outside that allows for a vacuum collection of lipoaspirate; and
c) a lid on the rigid container comprising a fluid logic,

wherein the fluid logic is controlled by a pinch cam which selectively pinches off tubing connections depending on the desired fluid configuration, and

wherein the fluid logic comprises a configuration such that when there is a need to open the canister, the adipose tissue collection and filtration component and lid remain in connection and closed to the atmosphere.

In some embodiments of the invention device, optionally in combination with one or more embodiments of invention disclosed herein, the entire assembly is pre-assembled and disposable.

In some embodiments of the invention device, optionally in combination with one or more embodiments of invention disclosed herein, the entire assembly is made entirely of plastic.

In some embodiments of the invention device, optionally in combination with one or more embodiments of invention disclosed herein, the fluid connections/logic is controlled by the product itself as opposed to humans needing to make and adjust connections.

In some embodiments of the invention device, optionally in combination with one or more embodiments of invention disclosed herein, the lid comprises a knob,

wherein the pinch cam comprises pitch valves, and

wherein turning of the knob causes the pinch valves to open and/or close in accordance with the fluid logic diagram.

In some embodiments of the invention device, optionally in combination with one or more embodiments of invention disclosed herein, the fluid logic comprises a plurality of ports and positions related to each other in a way to effect adipose tissue collection and purification.

In some embodiments of the invention device, optionally in combination with one or more embodiments of invention disclosed herein, the position comprises a harvest position, a wash position, and an off position, wherein:

in the “harvest” position, the fluid logic would be such that a vacuum source via a vacuum port would be pulling tissue from a patient port and into the adipose tissue collection and filtration component, in the meanwhile pulling fluid out of a “drain” port;

in the wash position, the drain would be closed off so the adipose tissue collection and filtration component is filled with fluid; and

in the off position, all of the pinch valves are closed and an “eccentric” cam is used to eject the lid off to open it and gain easy access to the adipose tissue collection and filtration component inside in the event that some of the tissue is stuck in the adipose tissue collection and filtration component to squeeze out the stuck tissue.

In some embodiments of the invention device, optionally in combination with one or more embodiments of invention disclosed herein, the adipose tissue collection and filtration component is a Puregraft™ bag.

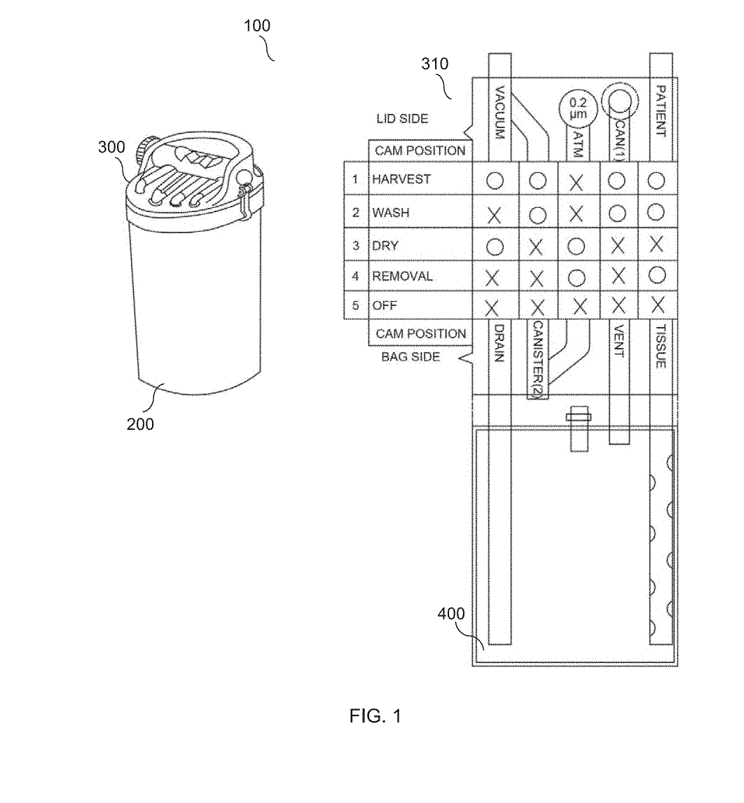

Exemplary embodiments of the invention are shown in FIGS. 1-3. Based on the current “Puregraft Vacuum lid” we were able to conceive and reduce to practice a device that wouldn't suffer from any of the above issues. The device would use Puregraft filtration on the inside, and would have a rigid container on the outside that would allow for a vacuum collection of lipoaspirate, and the entire assembly would be pre-assembled, disposable, and made entirely of plastic. The fluid connections/logic would be controlled by the product itself as opposed to humans needing to make and adjust connections.

In the current embodiment that has been prototyped, this fluid logic is controlled by a “pinch cam” which selectively pinches off tubing connections depending on the desired fluid configuration. Please see FIG. 1 for the fluid logic diagram, which is a core part of the invention. In this concept, the user would turn a knob, which opens and closes the pinch valves in accordance with the fluid logic diagram. As used herein, a pinch cam is meant to be a way to valve the fluid logic. Generically, any valves would work (i.e. ball valve, pinch valve, etc.)

For example, in the “harvest” position, the fluid logic would be such that the vacuum source (via the “vacuum” port) would be pulling tissue from the “patient” port and into the PG bag, while also pulling fluid out of the “drain” port. But in the “wash” position, the drain would be closed off so the bag could be filled with fluid. All of these connections and settings are subject to change, but the general idea of using a simple knob to control such complex fluid connections may be novel in this application.

In the “off” position, all of the valves would be closed and an “eccentric” cam would help to eject the lid off to open it and gain easy access to the bag inside in the event that some of the tissue was stuck in the bag and needed to be “squeezed” out, like other Puregraft bags, which has been effective. Note that even if the canister were to be opened in this manner, the PG bag and lid would still be in connection and “closed” to the atmosphere from a sterility point of view.

### Adipose Tissue Harvesting, Collection and Purification, and Application

In another aspect of the present invention, it is provided a method of adipose tissue collection and purification, comprising:

providing a device comprising
a) an adipose tissue collection and filtration component on the inside;
b) a rigid container (“canister”) on the outside that allows for a vacuum collection of lipoaspirate; and
c) a lid on the rigid container comprising a fluid logic, wherein the fluid logic is controlled by a pinch cam which selectively pinches off tubing connections depending on the desired fluid configuration, and wherein the fluid logic comprises a configuration such that when there is a need to open the canister, the adipose tissue collection and filtration component and lid remain in connection and closed to the atmosphere, and

performing adipose tissue collection and purification using the device so as to harvest and purify an amount of adipose tissue in a manner that the adipose tissue collection and filtration component and lid remain in connection and closed to the atmosphere.

In some embodiments of the invention method, optionally in combination with one or more embodiments of invention disclosed herein, the entire assembly of the device is pre-assembled and disposable.

In some embodiments of the invention method, optionally in combination with one or more embodiments of invention disclosed herein, the entire assembly of the device is made entirely of plastic.

In some embodiments of the invention method, optionally in combination with one or more embodiments of invention disclosed herein, the fluid connections/logic is controlled by the product itself as opposed to humans needing to make and adjust connections.

In some embodiments of the invention method, optionally in combination with one or more embodiments of invention disclosed herein, the lid comprises a knob,

wherein the pinch cam comprises pitch valves, and

wherein turning of the knob causes the pinch valves to open and/or close in accordance with the fluid logic diagram.

In some embodiments of the invention method, optionally in combination with one or more embodiments of invention disclosed herein, the fluid logic comprises a plurality of ports and positions related to each other in a way to effect adipose tissue collection and purification.

In some embodiments of the invention method, optionally in combination with one or more embodiments of invention disclosed herein, the position comprises a harvest position, a wash position, and an off position, wherein:

in the “harvest” position, the fluid logic would be such that a vacuum source via a vacuum port would be pulling tissue from a patient port and into the adipose tissue collection and filtration component, in the meanwhile pulling fluid out of a “drain” port;

in the wash position, the drain would be closed off so the adipose tissue collection and filtration component is filled with fluid; and

in the off position, all of the pinch valves are closed and an “eccentric” cam is used to eject the lid off to open it and gain easy access to the adipose tissue collection and filtration component inside in the event that some of the tissue is stuck in the adipose tissue collection and filtration component to squeeze out the stuck tissue.

In some embodiments of the invention method, optionally in combination with one or more embodiments of invention disclosed herein, the adipose tissue collection and filtration component is a Puregraft™ bag.

A brief description of the invention device as shown in FIGS. 1-3 is provided below.
a. Open box that contains PG canister+Lid+bag assembly and set onto mayo stand (sterile field)
b. Connect “Vacuum” port to preferred vacuum source an “Patient” port to preferred lipo cannula. Loop both tubes through the base clips (not pictured) to prevent tipping. Optional: clamp canister base to mayo stand.
c. With device in “Harvest” position, perform liposuction (extra fluid will drain during).
d. Adjust to “Wash” position and dip cannula into clean saline and suck in 1:1 ratio of saline to fat.
e. Adjust to “dry” to stop harvesting and suck out all of the waste fluid. Repeat 4 and 5 if desired.
f. Turn off lipo machine, and adjust device to “removal” position and extract fat via a catheter syringe out of the catheter tip port.
g. If all tissue cannot be removed, adjust to “off” position to lift lid. Remove PG bag.
h. Use traditional PG methods (hang and squeegee) to get all remaining tissue out of syringe port.
i. Dispose of entire PG system when finished.

### Method of Use

In a further aspect of the present invention, it is provided a method of using the device of invention, the adipose tissue collection and purification device of invention and/or the system of invention. In some embodiments, the use includes biomedical and cosmetic applications on a subject. Cosmetic applications can be, for example, organ reshaping or augmentation. Biomedical applications can be, for example, tissue grafting, and cell therapy or tissue regenerative therapies.

### EXAMPLES

### Example 1. Adipose Tissue Collection and Purification

FIGS. 1-3 describe embodiments of invention device and use of the invention device to collect and purified a volume of adipose tissue.

In FIG. 1, an invention device 100 includes a canister 200 having a lid 300. Lid 300 includes a liquid logic diagram 310 connected to a Puregraft™ bag 400. The liquid logic diagram includes cam positions marked from top down as 1) Harvest, 2) Wash, 3) Dry, 4) Removal, and O) Off; on the lid side—left to right, “Vacuum” (two ports joined as “Y”), “0.2 μm/ATM” “Can(1)”—canister 1, and “Patient′”; and on the bag side—left to right, which corresponds one-by-one with the lid side markings, “Drain”, “Canister (2)” joining the middle port as “Y”, “Vent”, and on the far right, “Tissue”. The “Tissue” tubing connects with PG Bag 400, which allows adipose tissue from patient to enter the bag and washed and the resultant waste liquid to drain from the bag with the assistance vacuum. As used herein, the label “ATM” refers to atmospheric pressure, and the other label “0.2 um” refers to a 0.2 micrometer pore size vent filter, common in blood processing. This port allows atmospheric pressure to enter the canister to allow one to squeeze the bag dry, meanwhile the contents of the bag remain closed to the environment.

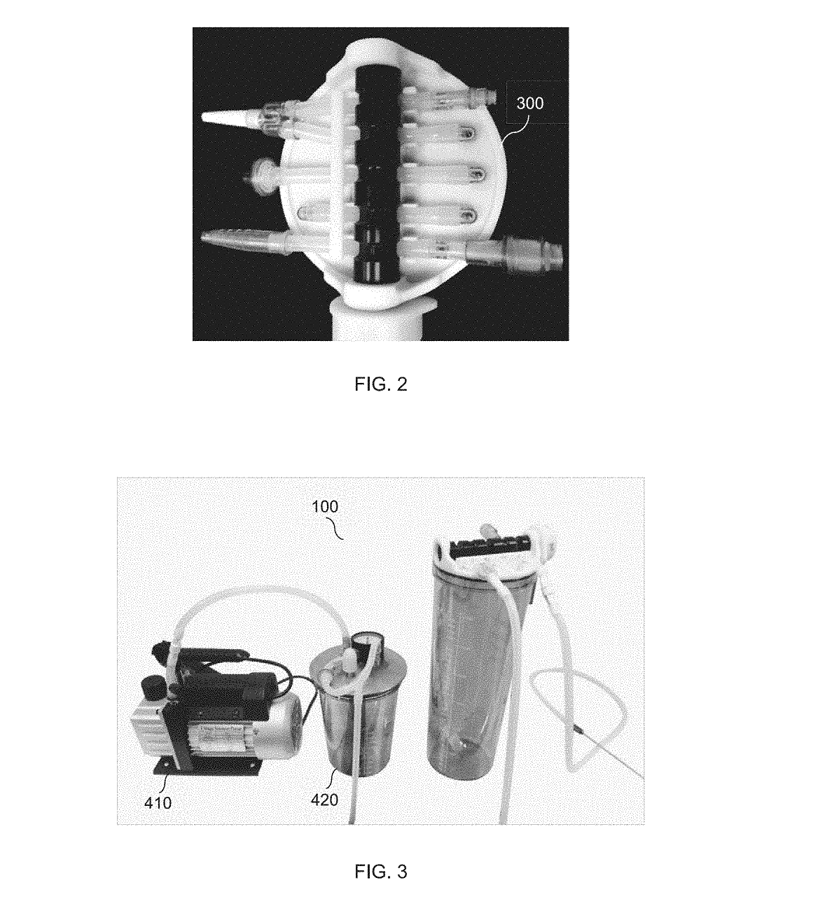

FIG. 2 is a photographic image of a 3D-printed/machined prototype of lid 300 with the liquid logic diagram 310.

FIG. 3 shows an invention device 100 being tested along with a pump 410 (left) and catch canister 420 (center).

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The teachings of the references, including patents and patent related documents, cited herein are incorporated herein in their entirety to the extent not inconsistent with the teachings herein.

We claim:

1. A device for adipose tissue collection and purification, comprising:
a) an adipose tissue collection and filtration component on the inside;
b) a rigid container (“canister”) on the outside that allows for a vacuum collection of lipoaspirate; and
c) a lid on the rigid container comprising a fluid logic,
wherein the fluid logic is controlled by a pinch cam which selectively pinches off tubing connections depending on the desired fluid configuration, and
wherein the fluid logic comprises a configuration such that when there is a need to open the canister, the adipose tissue collection and filtration component and lid remain in connection and closed to the atmosphere.

2. The device of claim 1, wherein the entire assembly is pre-assembled and disposable.

3. The device of claim 1, wherein the entire assembly is made entirely of plastic.

4. The device of claim 1, wherein the fluid connections/logic is controlled by the product itself as opposed to humans needing to make and adjust connections.

5. The device of claim 1, wherein the lid comprises a knob,
wherein the pinch cam comprises pitch valves, and
wherein turning of the knob causes the pinch valves to open and/or close in accordance with the fluid logic diagram.

6. The device of claim 1, wherein the fluid logic comprises a plurality of ports and positions related to each other in a way to effect adipose tissue collection and purification.

7. The device of claim 6, wherein the position comprises a harvest position, a wash position, and an off position, wherein:
in the “harvest” position, the fluid logic would be such that a vacuum source via a vacuum port would be pulling tissue from a patient port and into the adipose tissue collection and filtration component, in the meanwhile pulling fluid out of a “drain” port;
in the wash position, the drain would be closed off so the adipose tissue collection and filtration component is filled with fluid; and
in the off position, all of the pinch valves are closed and an “eccentric” cam is used to eject the lid off to open it and gain easy access to the adipose tissue collection and filtration component inside in the event that some of the tissue is stuck in the adipose tissue collection and filtration component to squeeze out the stuck tissue.

8. The device according to claim 7, wherein the adipose tissue collection and filtration component is a Puregraft™ bag.

9. A method of adipose tissue collection and purification, comprising:
providing a device comprising
a) an adipose tissue collection and filtration component on the inside;
b) a rigid container (“canister”) on the outside that allows for a vacuum collection of lipoaspirate; and
c) a lid on the rigid container comprising a fluid logic, wherein the fluid logic is controlled by a pinch cam which selectively pinches off tubing connections depending on the desired fluid configuration, and wherein the fluid logic comprises a configuration such that when there is a need to open the canister, the adipose tissue collection and filtration component and lid remain in connection and closed to the atmosphere, and
performing adipose tissue collection and purification using the device so as to harvest and purify an amount of adipose tissue in a manner that the adipose tissue collection and filtration component and lid remain in connection and closed to the atmosphere.

10. The method of claim 9, wherein the entire assembly of the device is pre-assembled and disposable.

11. The method of claim 9, wherein the entire assembly of the device is made entirely of plastic.

12. The method of claim 9, wherein the fluid connections/logic is controlled by the product itself as opposed to humans needing to make and adjust connections.

13. The method of claim 9, wherein the lid comprises a knob,
wherein the pinch cam comprises pitch valves, and
wherein turning of the knob causes the pinch valves to open and/or close in accordance with the fluid logic diagram.

14. The method of claim 9, wherein the fluid logic comprises a plurality of ports and positions related to each other in a way to effect adipose tissue collection and purification.

15. The method of claim 14, wherein the position comprises a harvest position, a wash position, and an off position, wherein:
in the “harvest” position, the fluid logic would be such that a vacuum source via a vacuum port would be pulling tissue from a patient port and into the adipose tissue collection and filtration component, in the meanwhile pulling fluid out of a “drain” port;
in the wash position, the drain would be closed off so the adipose tissue collection and filtration component is filled with fluid; and
in the off position, all of the pinch valves are closed and an “eccentric” cam is used to eject the lid off to open it and gain easy access to the adipose tissue collection and filtration component inside in the event that some of the tissue is stuck in the adipose tissue collection and filtration component to squeeze out the stuck tissue.

16. The method of claim 9, wherein the adipose tissue collection and filtration component is a Puregraft™ bag.
